(19) United States
(12) Patent Application Publication
(10) Pub. No.: US 2023/0109935 A1
(43) Pub. Date: Apr. 13, 2023

(54) APPARATUS FOR CLEANING ENVIRONMENTAL AIR USING ANTISEPTICS AND DISINFECTANTS

(71) Applicants: Shu-Ching HSU, Hsinchu City (TW); Li-Te CHIN, New Taipei City (TW)

(72) Inventors: Shu-Ching HSU, Hsinchu City (TW); Li-Te CHIN, New Taipei City (TW)

(21) Appl. No.: 17/953,468

(22) Filed: Sep. 27, 2022

Related U.S. Application Data
(60) Provisional application No. 63/248,645, filed on Sep. 27, 2021.

(51) Int. Cl. A61L 9/14

(52) U.S. Cl. CPC A61L 9/14; A61L 2101/06

(57) ABSTRACT

An apparatus for countercurrent exchange, including: a conduit, including: a side wall; an inner space; an air outlet disposed on a first end of the conduit; a liquid miniaturization device disposed in the inner space of the conduit near the air outlet, wherein the liquid miniaturization device is used to spray a liquid in a liquid direction; an air intake disposed on the side wall near a second end of the conduit; and a reservoir portion disposed in the inner space near the second end of the conduit for storing the liquid; a pneumatic conveyor connected with the air intake for introducing air into the conduit along an air flow direction; and a pumping device connected with the reservoir portion by a first tube, wherein the liquid direction is opposite to the air flow direction in the conduit.

### CROSS REFERENCE TO RELATED APPLICATION

This application claims the benefit of filing date of U.S. Provisional Application Serial No. 63/248,645, entitled “APPARATUS FOR CLEANING ENVIRONMENTAL AIR USING ANTISEPTICS AND DISINFECTANTS” filed Sep. 27, 2021 under 35 USC § 119(e)(1).

### BACKGROUND OF THE INVENTION

### 1. Field of the Invention

The invention relates to an integrated system of countercurrent exchange and involves the fields of human necessities, in particular for the use of hygiene (IPC A61).

### 2. Description of Related Art

The present invention relates to a novel system having a good efficiency for sanitization, in particular, a system incorporating the principles of countercurrent exchange to disinfect the ambient air for the purpose to remove or minimize fungi/mold. Today, it is well known that airborne transmissions of pathogens, non-pathogenic organisms, fragments of microbial cells, and byproducts of microbial metabolism, collectively referred to as “bioaerosols”, can all cause serious problems. It has been also concluded that there is a sufficient evidence of an association between the presence of “mold” (otherwise unspecified) in a damp indoor environment and asthma symptoms in sensitized asthmatic people (Stetzenbach et al., 2004). Among various microorganisms contributing bioaerosols, fungi are particular dangerous because they show a substantial tolerance to environmental conditions (Moldoveanu 2015; Kumar et al., 2021).

Bioaerosols are ubiquitous in our environment. Bioaerosols refer to all kinds of suspended particles or fragments derived from living organisms, including intact microorganisms (such as viruses, bacteria and fungi), metabolites and toxins produced by microorganisms, or any other suspended particulates generated from animals, plants or microorganisms. Exposure to bioaerosols may cause health hazards like infection, allergy, and toxic reactions, thus represents a very important environmental problem. A higher risk of exposure to biological hazards may due to the characteristics of the respective background, such as health care workers, cleaning team members, livestock or agriculture, forestry and fishery-related workers, miners, building renovation staff, nursery personnel, etc. In contrast to viruses, bacteria and fungi will grow, often to an alarming extent, on building materials if moisture is available. Background levels of airborne fungi change frequently inside buildings as a result of human activity. It has been documented that building conditions that allow excessive growth of bacteria or fungi can lead to occupants developing various specific medical symptoms or other complaints (Moldoveanu 2015).

Bioaerosols are ubiquitous in our environment. Bioaerosols refer to all kinds of However, due to the complex types of bioaerosols and the lack of appropriate standardized sanitization methods, the overall risk of bioaerosols, in particular indoor environments, is still high. In view of this, this invention combined hypochlorous acid (HClO)-based disinfectants and the principle of countercurrent exchange (CCE) for three dimensional indoor disfection we aslo evaluated the effectiveness of different hypochlorous acid (HClO) formulations in some example microorganisms.

Alcohol and aldehyde products, chlorine compounds, quaternary ammonium compounds, oxygen-containing chemicals, and enzymes are commonly used acquainted antiseptics and disinfectants which are exhibiting robust growth worldwide, especially during the period of pandemic. Nevertheless, most if not all of they are claimed to be effective against infectious agents, such as viruses, on surfaces. But in the real world scenario, biological infectious catastrophe is not only related and limited to a contaminated surface or two-dimensional route of spreading, airborne or three-dimensional transmission can be mediated by pathogen-containing large droplets emitted by coughing and/or sneezing that may reach an uninfected subject. Furthermore, inhalation of infectious aerosols or “droplet nuclei” smaller than 5 mm at a distance of >1 to 2 m away from the infected individual could acquire diseases as well (Eissenberg et al., 2020; Wang et al., 2021). Recently, several scientific reports have hypothesized that the presence of air pollutants — together with certain climatic conditions — might mean a long permanence of the viral particles in the air, which — in turn — could promote the indirect diffusion of the SARS-CoV-2 (Frontera et al., 2020; Yao et al., 2020). Together, those findings have strongly suggested that pathogens like the SARS-CoV-2 can be spreading through the air and that the transmission dynamics of COVID-19 could be due to the associations between particulate matter (PM) and the virus. In this same line, effective three-dimensional air disinfection/sanitization is critical to minimize the capacity for efficient human transmission of airborne pathogens.

In order to cope with the fast-mutating microorganisms which may be occurring in a more aggressive pattern by the association with air pollutants, only three established room-based technologies are available: mechanical ventilation, portable room air cleaners and upper room germicidal UV (GUV) air disinfection. For effective air disinfection by the mean of ventilation, a 6 to 12 room air changes per hour is recommended by the US Department of Health and Human Services Centers for Disease Control and Prevention (Guidelines for Environmental Infection Control in Health-Care Facilities, 2003). This requires mechanical ventilation systems to be designed and operated for high-flow rates—but at high operating costs when intake air must be heated or cooled and dehumidified. On the other hand, portable room air cleaners can be effective, but performance is limited by their clean air delivery rate relative to room volume.

Although in clinical settings, the UV-C (180-280 nm)-based commercially available upper-room GUV air disinfection with an effective rate of air mixing has been shown to reduce airborne tuberculosis transmission by 80%, i.e., equivalent to adding 24 room air changes per hour (Mphaphlele et al., 2015), damages caused by UV-C radiation are not selective to the pathogens, often in ways highly unfavorable to humans as well and thus causing severe burns of the skin and eye injuries (photokeratitis).

Similar to the UV-C radiation, toxic exposure of cleaning products to humans, animals and the environment is resulting from efforts to address the contagious pathogens with current sanitizers and disinfectants. By roaming and searching the US Environmental Protection Agency “N” list (https://www.epa.gov/pesticide-registration/list-n-disinfectants b-use-against-sars-cov-2) or the products’ Safety Data Sheet, we found that many of the products include chemicals that are harmful, e.g., quaternary ammonium compounds, bleach, hydrogen peroxide, ethyl alcohol or aldehyde. The most used household agent for the purpose of disinfection is bleach or hypochlorite solution, which contains 3-16% of sodium hypochlorite (NaOCl), its toxicity is not high, but may still cause corrosive damage after repetitive mild to moderate exposures. However, if the concentration is higher than 50 ppm, further use can cause skin irritation and even skin corrosion associated with other conditions.

The fumes from the bleach can also cause migraines, muscle weakness, abdominal discomfort, esophageal perforation, nausea and damage the nervous system. Fumes from the bleach can also linger indoors that are poorly ventilated; therefore the air then becomes polluted, endangering the health of everyone in the house.

On the contrary, hypochlorous acid (HClO), although having strong oxidizing power, is very low impact to the environment. The chemical structure of hypochlorous acid is difficult to be changed by heating or other physical factors; therefore, possible material damage is reduced. In practice, hypochlorous acid has been successfully applied to prevent fruit rotting, to clean wounds and to disinfect of surgical traumas. Moreover, slightly acidic hypochlorous acid waters have been shown to inactivate influenza virus and coronavirus (Miyaoka et al., 2021). A recent publication has indicated that 200 ppm of hypochlorous acid water has a good virus inactivation capability towards human SARS-CoV-2 (Block & Rowan 2020).

Because the airborne nature of many infectious diseases, it is tempting to expand that, if we are capable to disinfect a two-dimensional surface in an environmentally friendly way by using hypochlorous acid, it must have some validity in the three-dimensional practice. Although using hypochlorous acid water in our efforts to sanitize the environment are proving not poisonous, among its advantages of like powerful, easy to use without dilution and the disinfection can be achieved in seconds, there are disadvantage like short shelf life, sensitive to UV radiation and sunlight exposure. It will be reduced to water and salt within two hours of solarization, so the ideal conditions for disinfection and sterilization with hypochlorous acid solution should be closed and protected from light.

Countercurrent exchange (CCE) occurs in nature and is a widely used biological mechanism. Based on the principles of physics, a configuration of spatial arrangement is organized to achieve a greater efficiency of the mutual exchange; simultaneously the energy consumption is reduced. The property or substance is exchanged between two fluids providing that (1) the two fluids flow in close proximity to each other, and (2) the fluids flow in opposite directions (FIG. 1A). The primary purpose of CCE is to maintain a concentration gradient between the two fluids in order to maximize movement from one fluid to the other. Many organisms have CCE systems, such as: the gas exchange of fish gills, the heat conservation in the extremities of aquatic animals, the water preservation in the nose of desert rodents and so on.

In recent years, the world is constantly facing newly emergent pathogens which we have little or no natural defense against them, and thus the general population is vulnerable. Despite the knowledge accumulating on the effectiveness of HClO in disinfection and sanitization, the faint chlorine scent generated from spraying the solution (even in the form of dry microfine steam) and the limitation to two-dimensional application making the use of HClO still open to further development beyond its established safety. To combat various airborne diseases with HClO further than surface disinfection, the present invention applied the CCE principle by fulfilling its two prerequisites for attacking pathogens is in the air we live in. The novel configuration superimposes the two opposite flow, i.e., air and HClO in a single CCE conduit (FIG. 1B). It is clear to a person having ordinary skill in the art that the cross section of the CCE conduit is not restricted to a rectangular form nor an oblong shape, an artisan’s knowledge may go to concave and convex depending on, e.g., increasing the flow rates of the fluids or the room where CCE takes place.

The miniaturized disinfectant/sterilization liquid containing HClO represents the downward direction in the CCE conduit. It is nature that the work is done on a falling object, i.e., disinfectant, by the gravitational force. There are also plenty of structures already known in the art to miniaturize liquid into droplets like a shower head, spray nozzles or tips, and an atomizer, etc. Work done by gravity ensures that the downward solution is limited in the conduit. On the one hand, the ambient air inhaled by a pneumatic conveyor, such as a fan, flows upwards against the disinfectant. Thus a CCE effect not only sufficiently mixes the disinfectant and the air mutually, maximizing interaction of two fluids with each other; but also preventing the disinfectant to spill to the environment (FIG. 2).

In light of the general knowledge of a skilled artisan, common non-compressor cooling systems employ hydro wall, waterfalls, fog or mist for evaporative cooling (swap coolers). By using the principle of CCE, the present invention, in addition to air disinfection inside the CCE conduit, also provides a conventional evaporative cooling effect. Moreover, the cooling effect can be further enhanced through the incorporation of a semiconductor thermal electric cooling module (thermoelectric cooling modules; refrigeration chips; a Peltier semiconductors) to reduce the temperature of the aqueous solution. Thermoelectric cooling module has the advantage of energy conversion because of its small size, light weight, no noise, i.e., without the transmission element, and thus with a higher reliability, longer life, and eases for temperature control. Compared with general compressors, it can be incorporated in the present invention because it can operate without refrigerant and thus environmentally friendly.

### SUMMARY OF THE INVENTION

The object of the present invention is to provide an apparatus for countercurrent exchange, comprising: a conduit, comprising: a side wall; an inner space; an air outlet disposed on a first end of the conduit; a liquid miniaturization device disposed in the inner space of the conduit near the air outlet, wherein the liquid miniaturization device is used to spray a liquid in a liquid direction; an air intake disposed on the side wall near a second end of the conduit; and a reservoir portion disposed in the inner space near the second end of the conduit for storing the liquid; a pneumatic conveyor connected with the air intake for introducing air into the conduit along an air flow direction; and a pumping device connected with the reservoir portion by a first tube, wherein the liquid direction is opposite to the air flow direction in the conduit.

In an embodiment of the present invention, the reservoir portion may be used to store the liquid sprayed by the liquid miniaturization device for recycling purpose.

In an embodiment of the present invention, the pumping device may be connected with the liquid miniaturization device by a second tube.

In an embodiment of the present invention, the air intake may be at a first angle ranging from 0° to 75° with respect to a longitudinal axis of the conduit.

In an embodiment of the present invention, the liquid may be a sterilization liquid or a disinfection liquid.

In a further embodiment of the present invention, the disinfection liquid may contain hypochlorous acid.

In yet another embodiment of the present invention, the pumping device may be used to pump the liquid from the reservoir portion to the liquid miniaturization device.

Another object of the present invention is to provide a process for sanitizing indoor air, comprising the following steps: (i) providing aforementioned apparatus; (ii) adding the liquid into the reservoir portion, wherein the liquid is a sterilization liquid or a disinfection liquid; (iii) activating the pneumatic conveyor to introduce air into the conduit through the air intake; (iv) activating the pumping device in order to transport the liquid to the liquid miniaturization device; and (v) activating the liquid miniaturization device to spray the liquid enabling a countercurrent exchange with the introduced air, wherein the introduced air exits the conduit through the air outlet.

In an embodiment of the present invention, steps (iii), (iv), and (v) of the aforementioned process may be performed in sequence or simultaneously.

In another embodiment of the present invention, mold, bacteria or virus in the indoor air may be removed or minimized after step (v).

Other objects, advantages, and novel features of the invention will become more apparent from the following detailed description when taken in conjunction with the accompanying drawings.

### BRIEF DESCRIPTION OF THE DRAWINGS

FIGS. 1 (A-B) A is a schematic diagram of the CCE principle. B represents the structure of the novel CCE conduit designed to interact the air and liquid inside.

FIG. 2 is a schematic diagram of a practical CCE conduit for the purpose of disinfection.

FIG. 3 denotes a time-dependent reduction in the average indoor concentration (CFU m-³) of viable culturable count of fungi by using a representative disinfectant disclosed in the invention.

### DETAILED DESCRIPTION OF THE PREFERRED EMBODIMENT

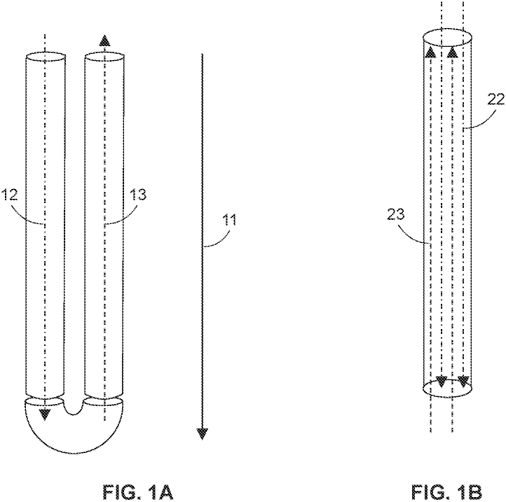

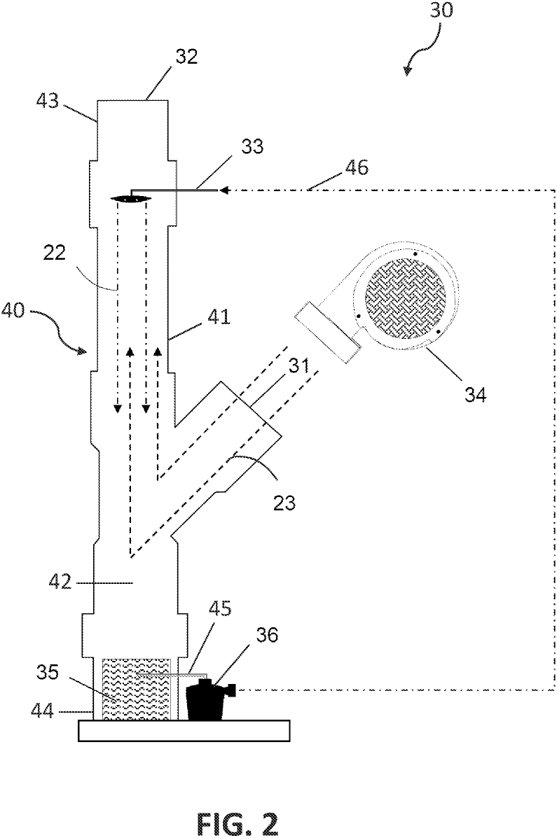

Referring to drawings in FIGS. 1-2, wherein similar components are identified by like reference numerals, there is seen in FIG. 1A, a conceptual view of the CCE principle with the direction of gravity 11, the flow direction of the first fluid 12, and the flow direction of the second fluid 13 provided. FIG. 1B represents a conceptual diagram of the present CCE apparatus with the liquid direction 22 and the air flow direction 23 provided.

FIG. 2 a view of the apparatus 30 in a first as used mode with the air intake 31 and air outlet 32 provided. The apparatus 30 includes a liquid miniaturization device 33, a pneumatic conveyor 34, a reservoir portion 35 and a pumping device 36. The pumping device 36 recycles the disinfectant from the reservoir portion to the liquid miniaturization device.

In one embodiment of the present invention, an apparatus 30 for countercurrent exchange is used, and the apparatus 30 comprises: a conduit 40, comprising: a side wall 41; an inner space 42; an air outlet 32 disposed on a first end 43 of the conduit 40; a liquid miniaturization device 33 disposed in the inner space 42 of the conduit 40 near the air outlet 32, wherein the liquid miniaturization device 33 is used to spray a liquid in a liquid direction 22; an air intake 31 disposed on the side wall near a second end 44 of the conduit 40; and a reservoir portion 35 disposed in the inner space 42 near the second end 44 of the conduit 40 for storing the liquid; a pneumatic conveyor 34 connected with the air intake 31 for introducing air into the conduit 40 along an air flow direction 23; and a pumping device 36 connected with the reservoir portion 35 by a first tube 45, wherein the liquid direction 22 is opposite to the air flow direction 23 in the conduit 40, wherein the pumping device 36 is connected with the liquid miniaturization device 33 by a second tube 46.

The present invention provides information of sanitizing or disinfecting indoor air for preferably removal of fungi. To this end, the air is mechanically inhaled into a conduit, whereby the air is fully mixed with hypochlorous acid (HClO)-containing disinfectant atomized with a liquid miniaturization device. To achieve a CCE effect, the path of air inside the conduit is upward and the path of atomized disinfectant is downward; to be directly interacted of air with disinfectant in a countercurrent configuration.

This invention also includes disinfectant compositions that contain, as the active ingredient, HClO, thereof in combination with an environmentally acceptable carriers or excipients. In preparing the composition of this invention, the HClO thereof is usually mixed with an excipient or diluted by an excipient. When the environmentally acceptable excipient serves as a diluent, it is preferable a liquid material, which acts as a vehicle, carrier, medium, or preservative for the active ingredient.

The following examples are hereby offeredto illustrate this invention and are not to be construed in any way as limiting the scope of the present invention.

### EXAMPLE 1: Mycological Examination

This embodiment uses a certain volume of air samples to be directly impacted on a medium suitable for fungal growth. Count of the total number of fungal colonies on the medium was used to calculate the total fungal concentration in one cubic meter of air after culturing at 25±1° C. for 5±2 days.

The medium for mold/fungi culture is Malt extract agar (MEA); each liter of MEA medium contains the following ingredients:
Malt (Maltose) 12.75 g
Dextrin (Dextrin) 2.75 g
Glycerol (glycerol) 2.35 g
Protein (Peptone) 0.78 g
Agar (Agar) 15.00 g

Dissolve the above components in 1 liter of distilled water pH4.7± 0.2 (25° C.), sterilized at 121° C. for 15 minutes. Subsequently placed in a water bath at about 50° C. to avoid condensation, cool to 45 to 50° C., supplemented with 0.05% chloramphenicol, and aliquot about 20 mL of medium in 90 × 15 mm Petri dishes and allowed them to solidify at room temperature.

Active air sampling procedures by impaction were performed as followed. Viable-culturable bioaerosol samples for fungi/mold were collected using a six-stage Andersen cascade impactor (Thermo Fisher Scientific, Waltham, MA, USA) with aerodynamic diameter cut points of 0.65, 1.1, 2.1, 3.3, 4.7, and >7.0 µm. The pump ensured a constant flow rate (28.3 L min⁻¹) through the impactor. A 90 × 15 mm Petri dish containing Malt extract Agar was placed on all the impactor stages. Impactors were disinfected of spraying 70% ethyl alcohol and wiping. The Petri dishes were incubated for at 25±1° C. for 5±2 days. Enumeration of fungi was conducted according to the previous artisans (Bogomolova & Kirtsideli 2008; Borrego et. al., 2010), and the concentration of fungi aerosol was measured in CFU (colony forming units)/m³ of air. The impactor was positioned in the middle of the room, 1.5 m above the floor and at least 0.5 m from the corners. The windows and doors of the rooms were closed during the sampling period.

### EXAMPLE 2: Formulations of Liquid Disinfectant

Disinfectants were formulated by preparing the following solution at pH 4.0.
Solution A: Hypochlorous acid water (hypochlorous acid; effective chlorine concentration 30 ppm);
Solution B: 30 ppm hypochlorous acid and 40 ppm sodium hypochloride;
Solution C: 30 ppm hypochlorous acid, 40 ppm sodium hypochloride and hydrogen peroxide 10 ppm
Solution D: 30 ppm hypochlorous acid, 40 ppm sodium hypochloride, 10 ppm hydrogen peroxide, and chloride salt (preferably calcium chloride or lithium choride)

### EXAMPLE 3: Outcome Measures

Two selected areas were used for evaluation where the environmental parameters are as followed:
1. The first studied area was a concrete floor office located in an urban region and air samples were taken indoors during the early summer season, when the average outdoor air temperature was about 35° C. and the indoor air-conditioning temperature was 24° C. The internal volume was 48.33 m³ and the relative humidity was 61%.
2. The second indoor environment was a standard 40’ dry cargo container with internal volume of 67.74 m³. The indoor temperature was 46° C. and the relative humidity was 75%.

The CCE disinfection and mycological sampling were performed with the door and/or windows closed. The ventilation of CCE conduit was set at 10 m³/min.

As currently there is no available international standard denoting acceptable fungal levels in indoor environments, to further evaluate the efficacy of CCE disinfection, indoor fungal counts were established. The present disclosure observes that the mean average concentration of the fungal aerosol in two indoor environments differed significantly, while it is practically indistinguishable between natural ventilation and air conditioning in the office area (Table 1). The mean average starting level of the fungal aerosol in the damp container with hot and elevated humidity was almost four times higher than the office area.

TABLE 1

Average indoor concentration (CFU m⁻³) of viable culturable count of fungi

| | CFU m⁻³ | N | SD |
|---|---|---|---|
| Office with natural ventilation | 414.62 | 9 | 85.20 |
| Office with air conditioning | 422.03 | 10 | 132.16 |
| Shipping container | 1571.17 | 7 | 310.75 |

N, number of sample; SD, standard deviation

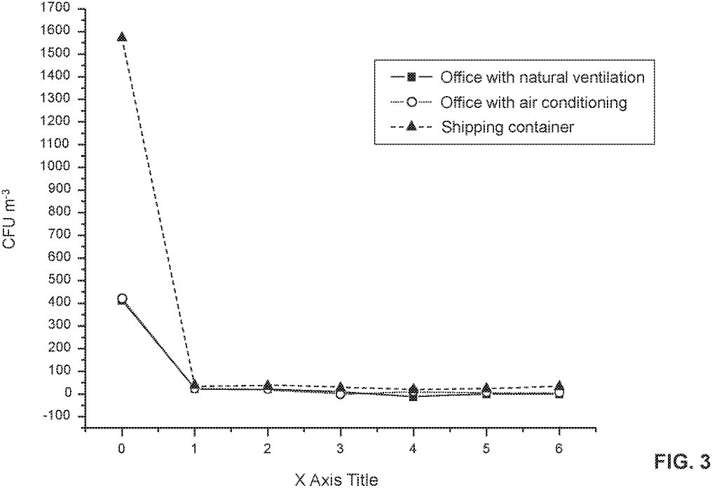

Using the average indoor concentrations as the reference initial fungal levels and to serve as a control, the invention compared the results with CCE disinfection every hour during six-hour cleaning period. Over the 6-hr duration of the investigation, operating the CCE disinfection for 1 hr caused the viable count of fungi to drop precipitately and remained at substantially low levels for the rest of 5 hr (FIG. 3). Furthermore, at the end of 1-hr treatment, there is no significant difference among four formulations of HOCl-containing disinfectants (Table 2), suggesting the efficiency for mycotic control in combination with CCE.

TABLE 2

Representative effect of CCE on average indoor concentration (CFU m⁻³) of fungi
CFU m⁻³

| | 0 hr | 1 hr | | | |
|---|---|---|---|---|---|
| | | A | B | C | D |
| Office with natural ventilation | 414.62 | 21.18 | 23.32 | 19.87 | 22.35 |
| Office with air conditioning | 422.03 | 25.32 | 24.18 | 23.10 | 24.67 |
| Shipping container | 1571.17 | 42.33 | 37.80 | 38.11 | 41.43 |

A, B, C and D represent four formulations of HOCl-containing disinfectants as disclosed.

A system encompasses a countercurrent exchange conduit and user-specific media for air circulation and cleaning. Within the conduit, a liquid miniaturization device (such as atomization device), liquid of antiseptic and/or disinfectant, and a pneumatic conveyor of said apparatus are localized in a proper direction, thereby forming sanitary cleaning equipments with good disinfection efficiency. The beneficial effect of the present invention is that it can disinfect and cool the ambient air without spilling the antiseptic or disinfectant out, which can achieve the functions of disinfection and sterilization and rapid heat dissipation at the same time, and economically and effectively achieve the purpose of environmental sanitation and cleaning control.

Although the present invention has been explained in relation to its preferred embodiment, it is to be understood that many other possible modifications and variations can be made without departing from the spirit and scope of the invention as hereinafter claimed.

What is claimed is:

1. An apparatus for countercurrent exchange, comprising:
a conduit, comprising:
a side wall;
an inner space;
an air outlet disposed on a first end of the conduit;
a liquid miniaturization device disposed in the inner space of the conduit near the air outlet, wherein the liquid miniaturization device is used to spray a liquid in a liquid direction;
an air intake disposed on the side wall near a second end of the conduit; and
a reservoir portion disposed in the inner space near the second end of the conduit for storing the liquid;
a pneumatic conveyor connected with the air intake for introducing air into the conduit along an air flow direction; and
a pumping device connected with the reservoir portion by a first tube,
wherein the liquid direction is opposite to the air flow direction in the conduit.

2. The apparatus of claim 1, wherein the pumping device is connected with the liquid miniaturization device by a second tube.

3. The apparatus of claim 1, wherein the air intake is at a first angle ranging from 0° to 75° with respect to a longitudinal axis of the conduit.

4. The apparatus of claim 1, wherein the liquid is a sterilization liquid or a disinfection liquid.

5. The apparatus of claim 4, wherein the disinfection liquid contains hypochlorous acid.

6. The apparatus of claim 1, wherein the pumping device is used to pump the liquid from the reservoir portion to the liquid miniaturization device.

7. A process for sanitizing indoor air, comprising the following steps:
(i) providing an apparatus for countercurrent exchange indoors, wherein the apparatus comprises:
a conduit, comprising:
a side wall;
an inner space;
an air outlet disposed on a first end of the conduit;
a liquid miniaturization device disposed in the inner space of the conduit near the air outlet, wherein the liquid miniaturization device is used to spray a liquid in a liquid direction;
an air intake disposed on the side wall near a second end of the conduit ; and
a reservoir portion disposed in the inner space near the second end of the conduit for storing the liquid;
a pneumatic conveyor connected with the air intake for introducing air into the conduit along an air flow direction; and
a pumping device connected with the reservoir portion by a first tube;
(ii) adding the liquid into the reservoir portion, wherein the liquid is a sterilization liquid or a disinfection liquid;
(iii) activating the pneumatic conveyor to introduce air into the conduit through the air intake;
(iv) activating the pumping device in order to transport the liquid to the liquid miniaturization device; and
(v) activating the liquid miniaturization device to spray the liquid enabling a countercurrent exchange with the introduced air,
wherein the introduced air exits the conduit through the air outlet.

8. The process of claim 7, wherein steps (iii), (iv), and (v) can be performed in sequence or simultaneously.

9. The process of claim 7, wherein the disinfection liquid contains hypochlorous acid.

10. The process of claim 7, wherein fungi, mold, bacteria or virus in the indoor air is removed or minimized after step (v).
